(19) United States
(12) Patent Application Publication
(10) Pub. No.: US 2020/0290954 A1
(43) Pub. Date: Sep. 17, 2020

(54) COMPOSITIONS OF OLIGOANILINES AND METHODS OF MAKING AND USING

(71) Applicant: Board of Regents, The University of Texas System, Austin, TX (US)

(72) Inventors: Alejandro Bugarin, Ft. Myers, FL (US); Gerardo Yepez, McAlester, OK (US); Anurag Noonikara-Poyil, Arlington, TX (US)

(21) Appl. No.: 16/817,965

(22) Filed: Mar. 13, 2020

Related U.S. Application Data
(60) Provisional application No. 62/819,210, filed on Mar. 15, 2019.

(51) Int. Cl. C07C 249/02; C07C 209/32; C07C 209/06

(52) U.S. Cl. CPC C07C 249/02; C07C 251/24; C07C 209/06; C07C 209/32

(57) ABSTRACT

Compositions of oligoanilines with higher purity, methods of making and using thereof, are provided. The compositions are produced in large scale with larger yield using simple purification techniques such as washing. Methods have been developed that allow large scale synthesis of oligoaniline compounds with the following benefits: (i) higher purity; (ii) larger yield of oligoaniline compounds; (iii) simple purification that does not require complicated techniques such as liquid chromatograph; (iv) lower cost; and (v) full characterization. The highly pure oligoaniline compositions can be used as reducing or oxidizing agent in a redox reaction. The oligoaniline compositions have colors and can be used as dyes, i.e. redox active dyes in a redox reaction, as intermediates for the development of conductive elastomers, or as catalysts.

### CROSS-REFERENCE TO RELATED APPLICATION

This application claims priority to and benefit of provisional application U.S. Ser. No. 62/819,210, filed Mar. 15, 2019, which is incorporated herein by reference in its entirety.

### FIELD OF THE INVENTION

The invention is generally directed to compositions of oligoanilines and the processes used to produce these compositions. Specifically, the present invention is directed to compositions of oligoanilines with high purity.

### BACKGROUND OF THE INVENTION

Polyanilines were first reported in 1862 (Letheby, et al., J. Chem. Soc., 15, 161). Since then, polyanilines have become a very important class of conductive and electronic polymers with several notable applications in electrochemistry and other related fields (MacDiarmid and Epstein, Faraday Discuss. Chem. Soc., 88:317-332 (1989)). Polyaniline properties have been extensively studied more than oligoanilines. Oligoanilines have found a wide variety of applications, including anticorrosive coatings (Chang, et al., ACS Appl. Mater. Interfaces, 5:1460 (2013); Yang, et al., J. Mater. Chem., 22:15845 (2012)), composites with graphene oxide (Lu, et al., ACS Appl. Mater. Interfaces, 9:4034 (2017)) and organic clay (Huang, et al., Polymer, 52:2391 (2011)), electrochemical functions (Lu, et al., Polym. Sci., Part A: Polym. Chem., 37:4295 (1999); Wang, et al., Macromolecules, 31:2702 (1998)), components of solar cells (Yildiz, et al., Adv. Funct. Mater., 18:3497 (2008)), tissue engineering constituents (Xie, et al., ACS Appl. Mater. Interfaces, 7:6772 (2015); Guo, et al., Biomacromolecules, 8:3025 (2007)), organic catalysts for oxidation reactions (Hirao, et al., J. Org. Chem., 63:7534 (1998)), and biodegradable conductive elastomers for biomedical applications, recently developed by Hong and Bugarin's groups (Xu, et al., Sci. Rep., 6:34451 (2016); Xu, et al., J. Biomed. Mater. Res. Part A, 104:2305 (2016)). Applications of oligoanilines are significantly lower when compared with those of polyanilines. The main limitation to their application has been in their synthesis, as they tend to polymerize.

Various strategies have been employed to synthesis oligoanilines, resulting in low yield, and/or a significant level of impurities in the final product. For example, small-scale synthesis of oligoanilines, such as N¹,N⁴-Bis(4-aminophenyl)-1,4-benzenediamine and N¹,N⁴-Bis(4-aminophenyl)-1,4-quinonediamine, have been previously reported.

N¹,N⁴-Bis(4-aminophenyl)-1,4-benzenediamine was previously synthesized using catalytic hydrogenation (yields not reported) was first reported in 1965 (Honzl, et al., Chem. Commun. (London), 0:440 (1965)). In 1989, Batich synthesized this oligoaniline via Schiff base chemistry and 79-(S_{N}Ar) reaction, albeit in low yields and impure products (Gebert, et al., Synth. Met., 29:371 (1989)). A small-scale synthesis of oligoanilines under high-pressure was reported by Brown et al., Synthesis, 16:2511 (2003). A similar method developed by Kulszewicz-Bajer gave access to a wide variety of oligoanilines derivatives of defined length (Rozalska, et al., New J. Chem., 28:1235 (2004); Rozalska, et al., Mol. Crypt. Liq. Crypt., 415:105 (2004)). A one-step synthesis using 4-chlorobenzenamine and p-phenylenediamine (yield not disclosed) is reported in Jetti, et al., Heterocycl. Chem., 50:E160 (2013).

With respect to N¹,N⁴-Bis(4-aminophenyl)-1,4-quinonediamine, Wie reported a one-pot synthesis of N¹,N⁴-Bis(4-aminophenyl)-1,4-quinonediamine (Wei, et al., Synth. Met., 84:289-291 (1997)), from p-phenylenediamine and aniline with 1 M HCl, Ethanol, (NH₄)₂S₂O₈, albeit in small-scale and low yield (Wei, et al., Tetrahedron Lett., 37:731 (1996)). Lastly, Durgaryan has also reported a synthetic method where p-phenylenediamine treated with K₂S₂O₈ (molar ratio of 4:1) in acetic acid at 15° C. yielded N¹,N⁴-Bis(4-aminophenyl)-1,4-quinonediamine as one of the products (Durgaryan, et al., Russ. J. Organ. Chem., 53:955 (2017)). Small scale synthesis of oligoaniline aniline tetramer, octamer, and 16-mer having a nitrogen atom at only one end has been reported in Surwade, et al., J. Am. Chem. Soc. 131:12528-12529 (2009); Surwade's reports a 59% crude yield of aniline octamer from its respective aniline tetramer and the pure yields for those isolated oligoaniline were not reported.

Major drawbacks remain in the prior art methods discussed above. First, isolation of products is a big-challenge for large-scale reactions because of the formation of byproducts and the presence of unreacted starting materials that need to be removed. Previous reports used liquid chromatography to purify oligoanilines in small scale, which is impossible for large-scale reactions. Second, none of the previous reports has documented reliable, reproducible, scalable, or a complete characterization data for oligoanilines. Third, there is no report of the synthesis of fully oxidized oligoanilines because of their tendency to polymerize during those reactions.

There is a need for simple synthesis methods for oligoanilines in large-scale with higher purity and larger yield.

It is therefore an object of the present invention to provide oligoaniline compositions with higher purity.

It is another object of the present invention to provide methods of making oligoaniline compositions with higher purity and larger yield.

It is yet another object of the present invention to provide methods of using oligoaniline compositions with higher purity.

### SUMMARY OF THE INVENTION

Oligoanilines, compositions of oligoanilines with higher purity, methods of making and using thereof, are provided. The compositions are produced in large scale with larger yield using simple purification techniques such as washing.

An oligoaniline having the structure of formula I is provided herein.

Also provided are compositions of oligoanilines with higher purity. The compositions include between 90% and 99% by weight the oligoaniline compound, preferably between 95% and 99% by weight the oligoaniline compound, more preferably between 98% and 99% by weight the oligoaniline compound as determined for example, by Elemental analysis. The oligoaniline compositions can include an oligoaniline which is a trimer, tetramer, pentamer, hexamer, heptamer, octamer, etc. A most preferred composition includes an oligoaniline which is a trimer, for example, oligoaniline compounds having the structure of Formula I, II, or III. Oligoanilines of different length, i.e. tetramer, pentamer, heptamers, octamers, etc. having the structures of Formula I, II, or III can be made by the disclosed method.

The aniline compositions in the most preferred embodiment include nitrogen atoms on both ends of the oligoaniline, i.e., using an aniline trimer as an example, the aniline trimers includes nitrogen atoms at both ends of the trimer.

Methods have been developed that allow large scale synthesis of compositions of oligoanilines with the following benefits: (i) higher purity; (ii) larger yield of oligoaniline compounds; (iii) simple purification that does not require complicated techniques such as liquid chromatograph; (iv) lower cost; and (v) full characterization. Exemplary methods include synthesizing a compound of formula III as disclosed herein, and subsequent reaction of the compound of formula III to provide compounds of formula II or formula I.

The highly pure oligoaniline compositions can be used as reducing or oxidizing agent in a redox reaction. The oligoaniline compositions have colors and can be used as redox active dyes in a redox reaction.

### DETAILED DESCRIPTION OF THE INVENTION

### I. Definitions

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied.

Numerical ranges disclosed in the present application of any type, disclose individually each possible number that such a range could reasonably encompass, as well as any sub-ranges and combinations of sub-ranges encompassed therein.

As used herein, the term “acyl group” refers to a formyl group, an acetyl group, a propionyl group, a butyryl group, an isobutyryl group, a valeryl group, an isovaleryl group, a benzoyl group or the like. Although the number of carbon atoms in the acyl group is not restricted, the number of carbon atoms generally ranges 1 to 20, preferably 1 to 8.

As used herein, the term “alkyl group” refers to univalent groups derived from alkanes by removal of a hydrogen atom from any carbon atom. Alkanes represent saturated hydrocarbons, including those that are cyclic (either monocyclic or polycyclic). Alkyl groups can be linear, branched, or cyclic. Preferred alkyl groups have one to 30 carbon atoms, i.e., C₁-C₃₀ alkyl. In some forms, a C₁-C₃₀ alkyl can be a linear C₁-C₃₀ alkyl, a branched C₁-C₃₀ alkyl, a cyclic C₁-C₃₀ alkyl, a linear or branched C₁-C₃₀ alkyl, a linear or cyclic C₁-C₃₀ alkyl, a branched or cyclic C₁-C₃₀ alkyl, or a linear, branched, or cyclic C₁-C₃₀ alkyl.

As used herein, the term “amide group” refers to —C(O)NHQ, —NHC(O)Q, —C(O)NQQ′, or —NQC(O)Q′. Q and Q′, respectively, represent an alkyl group, an alkenyl group, or an acryl group, similar to those exemplified with respect to the monovalent hydrocarbon group mentioned above.

As used herein, the term “aryl group” refers to univalent groups derived from arenes by removal of a hydrogen atom from a ring atom. Arenes are monocyclic and polycyclic aromatic hydrocarbons. In polycyclic aryl groups, the rings may be attached together in a pendant manner or may be fused. Preferred aryl groups have six to 50 carbon atoms, i.e., C₆-C₅₀ aryl. In some forms, a C₆-050 aryl can be a branched C₆-050 aryl, a monocyclic C₆-C₅₀ aryl, a polycyclic C₆-C₅₀ aryl, a branched polycyclic C₆-C₅₀ aryl, a fused polycyclic C₆-C₅₀ aryl, or a branched fused polycyclic C₆-C₅₀ aryl.

As used herein, the term “dehydrating agent” refers to a chemical compound that dries or removes water from a substance.

As used herein, the term “dye”, “dye molecule”, or “redox active dye molecule” refers to a colored substance that undergoes a color change during a reduction/oxidation reaction (redox reaction). The terms “dye”, “dye molecule”, and “redox active dye molecule” are used interchangeably throughout the instant disclosure.

As used herein, the term “ester group” refers to —C(O)OQ or —OC(O)Q. Q is an alkyl group, an alkenyl group, or an acryl group, similar to those exemplified with respect to the monovalent hydrocarbon group mentioned above.

As used herein, the term “halogen” or “halogen atoms” refers to fluorine, chlorine, bromine, or iodine atoms.

As used herein, the term “large scale” refers to the amount of starting material in a chemical reaction of at least 2 grams

As used herein, the term “monovalent hydrocarbon group” refers to an alkyl group such as a methyl group, an ethyl group, a propyl group, a butyl group, a t-butyl group, a hexyl group, an octyl group, a decyl group or the like; a cycloalkyl group such as a cyclopentyl group, a cyclohexyl group or the like; a bicycloalkyl group such as a bicyclohexyl group or the like; an alkenyl group such as a vinyl group, a 1-propenyl group, a 2-propenyl group, an isopropenyl group, a 1-methyl-2-propenyl group, a 1, 2 or 3-butenyl group, a hexenyl group or the like; an aryl group such as a phenyl group, a xylyl group, a tolyl group, a biphenyl group, a naphthyl group or the like; and an aralkyl group such as a benzyl group, a phenylethyl group, a phenyl cyclohexyl group or the like. It should be noted that part or the whole of hydrogen atoms of these monovalent hydrocarbon groups may be substituted with a hydroxyl group, a halogen atom, an amino group, a silanol group, a thiol group, a carboxyl group, a sulfone group, a phosphate group, a phosphoric acid ester group, an ester group, a thioester group, an amide group, a nitro group, an organoxy group, an organoamino group, an organosilyl group, an organothio group, an acyl group, an alkyl group, a cycloalkyl group, a bicycloalkyl group, an alkenyl group, an aryl group, an aralkyl group or the like. Although the number of carbon atoms in the monovalent hydrocarbon group is not restricted, the number of carbon atoms generally ranges 1 to 20, preferably 1 to 8.

As used herein, the term “oligoanilines” or “oligoaniline compounds” refers to organic compounds that contain aniline subunits connected at the para position, from two aniline residues up to twenty aniline residues. The terms “oligoanilines” and “oligoaniline compounds” are used interchangeably throughout the instant disclosure.

As used herein, the term “organoamino group” refers to an alkylamino group such as a phenylamino group, a methylamino group, an ethylamino group, a propylamino group, a butylamino group, a pentylamino group, a hexylamino group, a heptylamino group, an octylamino group, a nonylamino group, a decylamino group, a laurylamino group or the like; a dialkylamino group such as a dimethylamino group, a diethylamino group, a dipropylamino group, a dibutylamine group, a dipentylamine group, a dihexylamino group, a diheptylamino group, an dioctylamino group, a dinonylamino group, a didecylamino group or the like; or a cyclohexylamino group, a morpholino group or the like. Although the number of carbon atoms in the organoamino group is not restricted, the number of carbon atoms generally ranges 1 to 20, preferably 1 to 8.

As used herein, the term “organosilyl group” refers to a trimethylsilyl group, a triethylsilyl group, a tripropylsilyl group, a tributylsilyl group, a tripentylsilyl group, a trihexylsilyl group, a pentyldimethylsilyl group, a hexyldimethylsilyl group, an octyldimethylsilyl group, a decyldimethylsilyl group or the like. Although the number of carbon atoms in the organosilyl group is not restricted, the number of carbon atoms generally ranges 1 to 20, preferably 1 to 8.

As used herein, the term “organothio group” refers to an alkylthio group such as a methylthio group, an ethylthio group, a propylthio group, a butylthio group, a pentylthio group, a hexylthio group, a heptylthio group, an octylthio group, a nonylthio group, a decylthio group, a laurylthio group or the like. Although the number of carbon atoms in the organothio group is not restricted, the number of carbon atoms generally ranges 1 to 20, preferably 1 to 8.

As used herein, the term “organoxy group” refers to an alkoxy group, an alkenyloxy group, an aryloxy group, or the like, in which the alkyl group, alkenyl group and aryl group thereof may be those mentioned with respect to the above monovalent hydrocarbon group. Although the number of carbon atoms in the organoxy group is not restricted, the number of carbon atoms generally ranges 1 to 20, preferably 1 to 8.

As used herein, the term “oxidizing agent” refers to a substance that has the ability to oxidize other substances by causing them to lose electrons.

As used herein, the term “phosphoric acid ester group” refers to P(O)(OQ)(OQ′). Q and Q′, respectively, represent an alkyl group, an alkenyl group, or an acryl group, which is similar to those exemplified with respect to the monovalent hydrocarbon group mentioned above.

As used herein, the term “purifying” refers to the process of removing impurities.

As used herein, the term “purity” refers to the amount of an oligoaniline compound in a composition. “Higher purity” as used in connection with oligoaniline compositions refers to a oligoaniline compositions which include at least 90% (by weight) of an oligoaniline compound contained in a composition.

As used herein, the term “reducing” refers to the gaining of electrons by one or more of the atoms involved in a chemical reaction.

As used herein, the term “reducing agent” refers to a substance that has the ability to reduce other substances by causing them to gain electrons.

As used herein, the term “oxidation-reduction reaction” or “redox reaction” refers to a chemical reaction in which the oxidation states of atoms are changed. The terms “oxidation-reduction reaction” or “redox reaction” are used interchangeably throughout the instant disclosure.

As used herein, the term “room temperature” refers to a temperature within the range between 20° C. and 25° C., inclusive.

As used herein, the term “substituted” refers to the chemical group or moiety contains one or more substituents replacing the hydrogen atoms in the chemical group or moiety. The substituents include but not limited to the afore-mentioned substituent groups with respect to the monovalent hydrocarbon group. In the afore-mentioned substituent groups, a cyclic moiety wherein substituent groups are mutually bonded together may be contained.

As used herein, the term “thioester group” refers to C(S)OQ or —OC(S)Q. Q is an alkyl group, an alkenyl group, or an acryl group, similar to those exemplified with respect to the monovalent hydrocarbon group mentioned above.

As used herein, the term “unsubstituted” means bonding of a hydrogen atom.

As used herein, the term “yield” or “percentage yield” refers to the amount of product obtained in a chemical reaction. The percentage yield serves to measure the effectiveness of a synthetic procedure. It is calculated by dividing the amount of the obtained desired product by the theoretical yield (the unit of measure for both must be the same):

The theoretical yield is the amount predicted by a stoichiometric calculation based on the number of moles of all reactants present. This calculation assumes that only one reaction occurs and that the limiting reactant reacts completely

“High yield” is used to refer to the amount of product obtained in a chemical reaction is at least 70% of the theoretical amount of product based on stoichiometric calculation.

### II. Compositions

An oligoaniline having a structure shown below is provided.

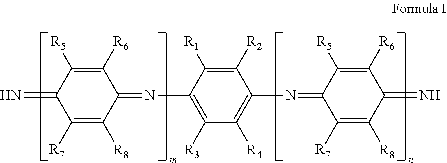

The Examples below synthesis of a new oxidized oligoaniline compounds having the structure of Formula I.

Compositions of oligoanilines with higher purity are also provided. For example, the compositions have between 90% and 99% by weight an oligoaniline compound, preferably between 95% and 99% by weight an oligoaniline compound, more preferably between 98% and 99% by weight an oligoaniline compound. In some embodiments, the disclosed oligoaniline compounds have the structure of Formula I, II, or III. The oligoaniline compounds disclosed herein can be fully characterized by NMR, Mass spectrometry, and Elemental analysis. In a preferred embodiment, the purity of the compositions is determined by elemental analysis.

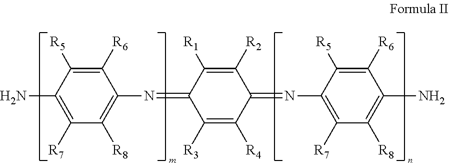

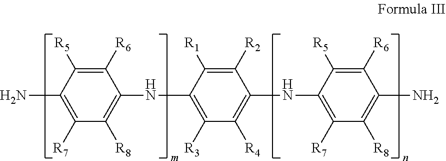

In preferred forms of compounds,

R₁ to R₈ independently represent a hydrogen atom, a halogen atom, a hydroxyl group, an amino group, a silanol group, a sulfonyl group, a thiol group, a carboxyl group, a phosphate group, a phosphoric acid ester group, an ester group, a carbonate ester group, a thioester group, an amide group, a nitro group, a monovalent hydrocarbon group, an organoxy group, an aminooxy group, a hydroxyamino group, an azo group, an organosilyl group, an organothio group, an alkyl group, an acryl group, an acyl group, an acryl group, or a sulfone group;

wherein R₁ to R₈ optionally form a ring with its neighboring R group, respectively.

In the most preferred embodiment, R₁ to R₈ are all hydrogen atoms.

In the oligoaniline compounds of Formula I, II, or III, m and n are independently an integer ≥1 provided that m+n≤20 is satisfied.

In some embodiments, m+n≤10. In some other embodiments, m+n≤8, preferably m+n≤6, more preferably m+n≤4. In the most preferred embodiment, the oligoaniline compounds of Formula I, II, or III are trimers in which m=1 and n=1.

The Examples below demonstrated for the first time the synthesis of highly pure compositions of oligoanilines in large scale.

### III. Methods of Making

Methods have been developed that allow large scale synthesis of compositions of oligoanilines with the following benefits: (i) higher purity; (ii) high yield of oligoaniline compounds; (iii) simple purification that does not require complicated techniques such as liquid chromatograph; (iv) lower cost; and (v) full characterization. The disclosed large scale synthesis of compositions of oligoanilines represents an easier access to oligoanilines and expedites the discovery of additional properties and/or applications of oligoanilines that are previously inaccessible.

The disclosed methods enable large scale (starting materials in grams) synthesis of oligoanilines, for example, the starting material is at least 2 grams, preferably at least 5 grams and more preferably, at least 8 grams. For example, the starting material can be present in an amount between, 10 and 100 grams, for example up to 10 grams, up to 20 grams, up to 30 grams, up to 40 grams etc.

The disclosed methods result in compositions of oligoanilines with higher purity. For example, the compositions have between 90% and 99%, preferably between 95% and 99%, more preferably between 98% and 99% by weight the oligoaniline.

The methods also result in high yield of the oligoaniline, preferably, a yield between 80% and 99%, between 85% and 99%, between 90% and 99%, and between 90% and 95% by weight. For example, the methods can result in a yield of 90%, 91%, 92%, 93%, 94%, etc. In some embodiments, the yield of compounds of Formula III is between 90% and 94%. In some embodiments, the yield of compounds of Formula II and/or compounds of Formula I is between 91% and 97%. In some embodiments, the yield of compounds of Formula A is between 79% and 82%.

The Examples below demonstrated for the first time the synthesis of highly pure compositions of oligoanilines in large scale. It is also the first demonstration of the synthesis of oxidized oligoanilines such as compounds of Formula I. It has been discovered that control of reaction temperatures, reagents (and reagent amounts) and the washing steps are critical to produce the highly pure compositions of oligoanilines.

Exemplary steps for making compositions of ologoanilines having formulas I, II and III are provided.

The methods include synthesizing a compound of formula III as disclosed herein, the subsequent reaction of the compound of formula III to provide compounds of formula II or formula I.

A. Methods of Making Composition of the Compound of Formula III

Compositions containing oligoaniline compound of Formula III are synthesized by:

(a) synthesizing a compound of Formula A, shown below;

(b) reducing the compound of Formula A to provide a compound of Formula III; and

(c) purifying the compound of Formula III to give the composition containing oligoaniline compound of Formula III.

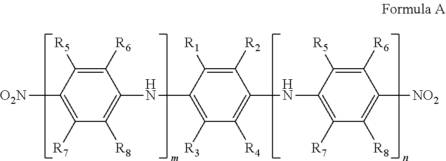

R₁ to R₈ independently represent a hydrogen atom, a halogen atom, a hydroxyl group, an amino group, a silanol group, a sulfonyl group, a thiol group, a carboxyl group, a phosphate group, a phosphoric acid ester group, an ester group, a carbonate ester group, a thioester group, an amide group, a nitro group, a monovalent hydrocarbon group, an organoxy group, an aminooxy group, a hydroxyamino group, an azo group, an organosilyl group, an organothio group, an alkyl group, an acryl group, an acyl group, an acryl group, or a sulfone group;

wherein R₁ to R₈ optionally form a ring with its neighboring R group, respectively; and

wherein m and n are independently an integer ≥1 provided that m+n≤20 is satisfied.

In some embodiments, m+n≤10. In some other embodiments, m+n≤8, preferably m+n≤6, more preferably m+n≤4. In a preferred embodiment, the oligoaniline compound of Formula A is N¹, N⁴-Bis(4-nitrophenyl)-1,4-benzenediamine, in which m=1 and n=1. In one embodiment, N¹, N⁴—Bis(4-nitrophenyl)-1,4-benzenediamine has a red color.

1. Synthesizing a Compound of Formula A

The method steps for synthesizing a compound of formula A include

(a1) mixing a compound of Formula 1 with a compound of Formula 2 and an amine of Formula 3 in a first solvent to provide a reaction mixture;

(a2) providing a reaction condition for the reaction mixture from a1 to form the compound of Formula A; and

(a3) purifying the compound of Formula A from the reaction mixture.

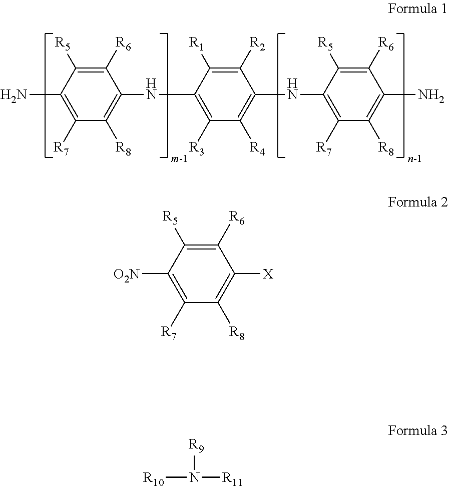

R₁ to R₈ independently represent a hydrogen atom, a halogen atom, a hydroxyl group, an amino group, a silanol group, a sulfonyl group, a thiol group, a carboxyl group, a phosphate group, a phosphoric acid ester group, an ester group, a carbonate ester group, a thioester group, an amide group, a nitro group, a monovalent hydrocarbon group, an organoxy group, an aminooxy group, a hydroxyamino group, an azo group, an organosilyl group, an organothio group, an alkyl group, an acryl group, an acyl group, an acryl group, or a sulfone group;

wherein R₁ to R₈ optionally form a ring with its neighboring R group, respectively,

wherein R₉ to R₁₁ independently represent a monovalent hydrocarbon group,

wherein X represents a halogen, and

wherein m and n are independently an integer ≥1 provided that m+n≤20 is satisfied.

In some embodiments, X represents a fluorine.

In some embodiments, m+n≤10. In some other embodiments, m+n≤8, preferably m+n≤6, more preferably m+n≤4. In a preferred embodiment, m+n=2 in which m=1, n=1.

In some embodiments, R₁ to R₈ are all hydrogen atoms. In a particularly preferred embodiment, the oligoaniline compounds of Formula 1 is p-phenylenediamine, in which m=1 and n=1; the compound of Formula 2 is 1-fluoro-4-nitrobenzene in which X is fluorine; and the amine of Formula 3 is trimethylamine, in which R₉ to R₁₁ are ethyl groups.

In some embodiments, the compound of Formula 1 is between 10 and 100 grams, between 10 and 80 grams, between 10 and 50 grams, and between 10 and 20 grams. In one embodiment, the compound of Formula 1 is about 12.3 grams. In some embodiments, the mole ratio of Formula 1:Formula 2 is between 1:1 and 1:10. Example mole ratios of Formula 1:Formula 2 include between about 1:1 and 1:10, between 1:1 and 1:5, and between 1:1 and 2.5. In one embodiment, the mole ratio of Formula 1:Formula 2 is 1:2.5. In some embodiments, the mole ratio of Formula 1:Formula 3 is between 1:1 and 1:10. Example mole ratios of Formula 1:Formula 3 include between about 1:1 and 1:10, between 1:1 and 1:5, and between 1:1 and 1:2. In one embodiment, the mole ratio of Formula 1:Formula 3 is 1:2.

In the most preferred embodiment, the compound of Formula 1 is about 12.3 grams, the compound of Formula 2 is about 40 grams, and the compound of Formula 3 is about 23 grams.

Example of solvents that can be used in step a1 include, but are not limited to dimethyl sulfoxide, methylene chloride, chloroform, tetrahydrofuran, acetone, dioxane, ethyl acetate, dimethylene carbonate, dimethyl formamide, methyl ethyl ketone, butyl acetate, butyl propionate, and diethyl carbonate. In a preferred embodiment, the solvent is dimethyl sulfoxide. In some embodiments, the amount of solvent used in step a1 is between 50 mL and 500 mL, between 100 mL and 500 mL, between 150 mL and 500 mL, between 200 mL and 500 mL, between 250 mL and 500 mL, between 300 mL and 500 mL, between 400 mL and 500 mL, between 50 mL and 100 mL, between 50 mL and 150 mL, between 50 mL and 200 mL, between 50 mL and 250 mL, between 50 mL and 300 mL, between 50 mL and 350 mL, between 50 mL and 400 mL, or between 50 mL and 450 mL.

In some embodiments, the concentration of the compound of Formula 1 is between 0.1 M and 10 M, between 0.2 M and 10 M, between 0.5 M and 10 M, between 1 M and 10 M, between 0.1 M and 5 M, between 0.2 M and 5 M, between 0.5 M and 5 M, between 1 M and 5 M, between 0.1 M and 2 M, between 0.2 M and 2 M, between 0.5 M and 2 M, or between 1 M and 2 M.

In some embodiments, the concentration of the compound of Formula 2 is between 0.1 M and 10 M, between 0.2 M and 10 M, between 0.5 M and 10 M, between 1 M and 10 M, between 0.1 M and 5 M, between 0.2 M and 5 M, between 0.5 M and 5 M, between 1 M and 5 M, between 0.1 M and 2 M, between 0.2 M and 2 M, between 0.5 M and 2 M, or between 1 M and 2 M. In some embodiments, the concentration of the compound of Formula 3 is between 0.1 M and 10 M, between 0.2 M and 10 M, between 0.5 M and 10 M, between 1 M and 10 M, between 0.1 M and 5 M, between 0.2 M and 5 M, between 0.5 M and 5 M, between 1 M and 5 M, between 0.1 M and 2 M, between 0.2 M and 2 M, between 0.5 M and 2 M, or between 1 M and 2 M.

Failure to use the concentration of compound of Formula 1, Formula 2, and/or Formula 3 disclosed herein can result in incomplete reaction and/or byproducts formation thus introduce impurities.

In some embodiments, step (a2) of the methods includes providing a reaction temperature for the reaction mixture between 80° C. and 200° C. and a reaction time between 1 and 100 hours. The reaction temperature is between 80° C. and 180° C., between 80° C. and 160° C., between 80° C. and 150° C., between 80° C. and 125° C., between 90° C. and 200° C., between 100° C. and 200° C., between 120° C. and 200° C., between 90° C. and 150° C., between 100° C. and 150° C., and between 120° C. and 150° C. In one embodiment, the reaction temperature is about 125° C.

The reaction time is within the range between 1 and 100 hours, between 10 and 100 hours, between 20 and 100 hours, between 30 and 100 hours, between 50 and 100 hours, between 70 and 100 hours, between 70 and 90 hours, and between 70 and 80 hours. In one embodiment, the reaction time is about 72 hours.

Following reaction in at the temperature and for the disclosed time, the oligoaniline of formula A is purified from the reaction mixture.

In some embodiments, the purification step (a3) of the methods includes:

(a3-1) collecting the compound of Formula A from the reaction mixture;

(a3-2) washing the compound of Formula A with a second solvent and water; and

(a3-3) washing the compound of Formula A with water.

The purification step of the methods does not require complicated purification techniques, such as liquid chromatograph. This is particularly desirable since it greatly simplifies the purification process and lowers the cost of synthesis. The compound of Formula A may be collected by any suitable means.

The washing steps (a3-2) and (a3-3) are to remove side products in the reaction mixture, which are soluble in water under acidic conditions. In a preferred embodiment, the temperature of the washing steps (a3-2) and (a3-3) should be less than 50° C. and greater than 0° C., and more preferably at room temperature.

(a3-1) Collection of the Compound of Formula a from the Reaction Mixture

In some embodiment, the solid is collected by filtration or centrifugation. In a particular embodiment, the reaction mixture is filtered using a 600 mL (40-60 C) frit sintered glass filter funnel equipped with grade 415 filter paper (qualitative, grade 415 was used (VWR®)).

(a3-2) Washing of Compound of Formula a with a Second Solvent and Water

The second solvent is preferably a mixed solvent including an acid and a solvent selected from dimethyl sulfoxide, methylene chloride, chloroform, tetrahydrofuran, acetone, dioxane, ethyl acetate, dimethylene carbonate, dimethyl formamide, methyl ethyl ketone, butyl acetate, butyl propionate, and diethyl carbonate. In a preferred embodiment, the solvent mixed with an acid is dimethyl sulfoxide.

The acid can be hydrochloric acid (HCl), nitric acid, hydroiodic acid, perchloric acid, chloric acid, sulfuric acid, hydrobromic acid, sulfurous acid, methanoic acid, phosphoric acid, nitrous acid, oxalic acid, methanoic acid, benzoic acid, acetic acid, formic acid, or hydrofluoric acid. In a preferred embodiment, the acid is HCl. In a particularly preferred embodiment, the mixed solvent includes HCl and dimethyl sulfoxide. The ratio of the acid to the third solvent is within the range between 1/1 (v/v) and 10/1 (v/v), between 1/1 (v/v) and 8/1 (v/v), between 1/1 (v/v) and 6/1 (v/v), between 1/1 (v/v) and 4/1 (v/v), between 1/1 (v/v) and 2/1 (v/v), between 4/1 (v/v) and 10/1 (v/v), between 6/1 (v/v) and 10/1 (v/v), and between 8/1 (v/v) and 10/1 (v/v). In one embodiment, the ratio of the acid to the third solvent is 2/1 (v/v).

In some embodiments, the concentration of the acid is between 2 M and 10 M, between 3 M and 10 M, between 4 M and 10 M, between 5 M and 10 M, between 2 M and 9 M, between 3 M and 9 M, between 4 M and 9 M, between 5 M and 9 M, between 2 M and 8 M, between 3 M and 8 M, between 4 M and 8 M, between 5 M and 8 M, between 2 M and 7 M, between 3 M and 7 M, between 4 M and 7 M, between 5 M and 7 M, between 2 M and 6 M, between 3 M and 6 M, between 4 M and 6 M, between 5 M and 6 M, between 4.5 M and 6 M, or between 4.5 M and 5.5 M. In some embodiments, the concentration of the acid is about 5 M. In a particular embodiment, the concentration of HCl is about 5 M. Failure to use the concentration of HCl disclosed herein can result in insufficient removal of impurities and/or undesirable polymerization of the compound of Formula A.

(a3-3) Washing the Compound of Formula a with Water

In some embodiments, following washing with the second solvent (preferably once), the collected compound is washed twice with water.

This washing process can be repeated at least 2 times, preferably at least 7 times. The optimum amount of the second solvent required to wash the solid will be dependent upon the amount of solid and the number of washes. In a preferred embodiment, this process is repeated for 7 times with a total of 700 mL of 5 M HCl/DMSO (2/1, v/v) solution and 1400 mL of deionized water to remove side products, which are soluble in water under acidic conditions.

If desired, additional washing of the collected compound of Formula A may be performed with water and an organic solvent after performing steps (a3-2) and (a3-3). The compound of Formula A may be further washed with deionized water 5 times (5×100 mL) and then with dichloromethane (DCM), 5 times (5×100 mL). Although it is not necessary to completely dry the compound of Formula A after washing, the washed compound of Formula A is dried by any suitable means. In some embodiments, the compound of Formula A is air-dried, dried in a vacuum oven, or using a dehydrating agent. In a preferred embodiment, the compound of Formula A is dried in a vacuum oven at a temperature of about 80° C.

2. Reducing the Compound of Formula A

The compound of Formula A can be reduced by:

(b1) mixing the compound of Formula A with an acid and a metal to provide a reaction mixture; and

(b2) providing reaction conditions for the reaction mixture to form the compound of Formula III.

(b1) Mixing the Compound of Formula a with an Acid and a Metal to Provide a Reaction Mixture

The acid can be hydrochloric acid (HCl), nitric acid, hydroiodic acid, perchloric acid, chloric acid, sulfuric acid, hydrobromic acid, sulfurous acid, methanoic acid, phosphoric acid, nitrous acid, oxalic acid, methanoic acid, benzoic acid, acetic acid, formic acid, or hydrofluoric acid. In a preferred embodiment, the acid is HCl.

In some embodiments, the concentration of the acid is between 2 M and 10 M, between 3 M and 10 M, between 4 M and 10 M, between 5 M and 10 M, between 2 M and 9 M, between 3 M and 9 M, between 4 M and 9 M, between 5 M and 9 M, between 2 M and 8 M, between 3 M and 8 M, between 4 M and 8 M, between 5 M and 8 M, between 2 M and 7 M, between 3 M and 7 M, between 4 M and 7 M, between 5 M and 7 M, between 2 M and 6 M, between 3 M and 6 M, between 4 M and 6 M, between 5 M and 6 M, between 4.5 M and 6 M, or between 4.5 M and 5.5 M. In some embodiments, the concentration of the acid is about 5 M. In a particular embodiment, the concentration of HCl is about 5 M.

The metal can be transition metal or post-transitional metal. In some embodiments, the metal is a transitional metal selected from Zinc, Iron, and Nickel. In some embodiments, the metal is a post-transitional metal selected from aluminum (Al), gallium (Ga), indium (In), thallium (Tl), Tin (Sn), lead (Pb) and bismuth (Bi). In a particularly preferred embodiment, the metal is Tin (Sn). In some embodiments, the amount of metal to the amount of compound of Formula A is at least 4 mol/mol, at least 5 mol/mol, at least 6 mol/mol, at least 7 mol/mol, or at least 8 mol/mol. In some embodiments, the amount of metal to the amount of compound of Formula A is between 4 mol/mol and 12 mole/mol, between 4 mol/mol and 11 mole/mol, between 4 mol/mol and 10 mole/mol, between 4 mol/mol and 9 mole/mol, between 4 mol/mol and 8 mole/mol, between 4 mol/mol and 7 mole/mol, between 4 mol/mol and 6 mole/mol, between 5 mol/mol and 12 mole/mol, between 5 mol/mol and 11 mole/mol, between 5 mol/mol and 10 mole/mol, between 5 mol/mol and 9 mole/mol, between 5 mol/mol and 8 mole/mol, between 5 mol/mol and 7 mole/mol, between 5 mol/mol and 6 mole/mol. In some embodiments, the amount of metal to the amount of compound of Formula A is about 5 mol/mol.

The amount of compound of Formula A used as starting material in step (b1) is between 10 and 100 grams, between 10 and 80 grams, between 10 and 50 grams, between 20 and 50 grams, between 30 and 50 grams, and between 20 and 40 grams. In one embodiment, the compound of Formula A is about 30 grams. In some embodiments, the mole ratio of Formula A:metal is between 1:1 and 1:10. Example mole ratios of Formula A:metal include between about 1:1 and 1:10, between 1:1 and 1:8, between 1:1 and 1:5, and between 1:2 and 1:5. In one embodiment, the mole ratio of Formula A:metal is 1:5. In the most preferred embodiment, the compound of Formula 1 is about 30 grams and the metal is about 50.8 grams.

(b2) Reaction Conditions for the Reaction Mixture to Form the Compound of Formula III

The reaction mixture provided by step (b1) is mixed at a temperature between 0° C. and 40° C. for a period between 0.1 and 10 hours to avoid overheating. In a particularly preferred embodiment, the reaction mixture is mixed at room temperature for a period of about 2 hours by stirring.

Following mixing of the reaction mixture at a temperature between 0° C. and 40° C. for a period between 0.1 and 10 hours, the reaction mixture is provided with a reaction temperature between 60° C. and 100° C. and a reaction time between 1 and 100 hours. The reaction temperature is within the range between 60° C. and 100° C., between 70° C. and 100° C., between 80° C. and 100° C., and between 90° C. and 100° C. In a preferred embodiment, the reaction temperature is about 90° C. Preferably, the reaction temperature is provided gradually by increasing the temperature from room temperature to the desired reaction temperature over a period between 1 and 60 minutes, between 1 and 30 minutes, between 1 and 20 minutes, between 10 and 60 minutes, and between 10 and 30 minutes. In a preferred embodiment, the reaction temperature is gradually increased from room temperature to about 90° C. over a period of about 20 minutes. The reaction time is within the range between 1 and 100 hours, between 1 and 90 hours, between 1 and 80 hours, between 1 and 70 hours, between 1 and 60 hours, between 1 and 50 hours, between 1 and 40 hours, between 1 and 30 hours, between 1 and 20 hours, and between 1 and 10 hours. In a preferred embodiment, the reaction time is about 5.5 hours.

The reagents and conditions disclosed above result in a compound of Formula III.

3. Purifying the Compound of Formula III

Purifying the compound of Formula III includes:

(c1) collecting the compound of Formula III from the reaction mixture after step b2, above; and

(c2) washing the collected compound of Formula III to obtain a composition containing the compound of Formula III.

The purification step (c) of the methods does not require complicated purification techniques, such as liquid chromatograph. This is particularly desirable since it greatly simplifies the purification process and lowers the cost of the synthesis. The compound of Formula III may be collected by any suitable means. In some embodiment, the solid is collected by filtration or centrifugation. In a particular embodiment, the reaction mixture is filtered using a 600 mL (40-60 C) frit sintered glass filter funnel equipped with grade 415 filter paper (qualitative, grade 415 was used (VWR®)).

The washing step (c2) is employed to remove excess metal and other impurities that can otherwise contaminate the composition containing compound of Formula III. In some embodiments, the washing step (c2) includes sub-steps:

(c2-1) washing the compound of Formula III obtained after step b2 with an acid to provide an acid-washed compound of Formula III;

(c2-2) mixing the acid-washed compound of Formula III with a base for a period between 1 and 100 minutes to provide a compound of Formula III/base mixture; and

(c2-3) collecting a composition containing the compound of Formula III from the compound of Formula III/base mixture.

Failure to perform steps (c2-1) and (c2-2) results in a mixture of compound of Formula III and unacceptable levels of impurities. In a preferred embodiment, the temperature of the washing step (c2) should be less than 50° C. and greater than 0° C., and more preferably at room temperature.

Useful acids for washing in step c2-1 include, but are not limited to hydrochloric acid (HCl), nitric acid, hydroiodic acid, perchloric acid, chloric acid, sulfuric acid, hydrobromic acid, sulfurous acid, methanoic acid, phosphoric acid, nitrous acid, oxalic acid, methanoic acid, benzoic acid, acetic acid, formic acid, or hydrofluoric acid. In a preferred embodiment, the acid is HCl.

Useful bases include sodium hydroxide (NaOH), lithinium hydroxide (LiOH), potassium hydroxide (KOH), rubidium hydroxide (RbOH), or cesium hydroxide (CsOH). In a preferred embodiment, the base is NaOH.

The acid-washed compound of Formula III is mixed with the base for a period between 1 and 100 minutes, between 10 and 90 minutes, between 20 and 90 minutes, between 30 and 90 minutes, between 10 and 80 minutes, between 10 and 60 minutes, between 10 and 40 minutes, between 20 and 40 minutes. In a preferred embodiment, the acid-washed compound of Formula III is mixed with the base for about 30 minutes by stirring. In some embodiments, the mixing step can quench the acid for full neutralization.

The composition containing the compound of Formula III may be collected by any suitable means. In some embodiment, the composition is collected by filtration or centrifugation. In a particular embodiment, the reaction mixture is filtered using a 600 mL (40-60 C) frit sintered glass filter funnel equipped with grade 415 filter paper (qualitative, grade 415 was used (VWR®)).

If desired, additional washing of the collected composition containing the compound of Formula III may be performed with water. In one embodiment, the compound of Formula III is further washed with deionized water for 10 times (10×100 mL) to neutralize the compound of Formula III. Although it is not necessary to completely dry the compound of Formula III after washing, in a preferred embodiment, the washed compound of Formula III is dried by any suitable means. In some embodiments, the compound of Formula III is air-dried, dried in a vacuum oven, or using a dehydrating agent. In a preferred embodiment, the compound of Formula III is dried in a vacuum oven at a temperature of about 80° C. for about 12 hours.

B. Methods of Making the Composition Containing Compound of Formula II

The compound of Formula II is made using the compound of Formula III as the starting material using the steps of:

(a) dissolving a compound of Formula III in a solvent to provide a reaction mixture;

(b) providing a reaction condition for the reaction mixture to form the compound of Formula II; and

(c) purifying the compound of Formula II from the reaction mixture to give the composition containing the compound of Formula II.

The purification step (c) of the methods does not require complicated purification techniques, such as liquid chromatograph. This is particularly desirable since it greatly simplifies the purification process and lowers the cost of synthesis.

The resulting composition have between 90% and 99% by weight the compound of Formula II, preferably between 95% and 99% by weight the compound of Formula II, more preferably between 98% and 99% by weight the compound of Formula II.

The methods result in the compound of Formula II with a yield between 80% and 99%, between 85% and 99%, between 90% and 99%, and between 90% and 95% by weight. In a preferred embodiment, the compound of Formula II has a yield between 91% and 96% by weight.

The amount of compound of Formula III used as starting material in step (a) is between 5 and 100 grams, between 5 and 80 grams, between 5 and 50 grams, between 5 and 20 grams, and between 5 and 10 grams. In one embodiment, the compound of Formula III used as starting material is about 8 grams.

Exemplary solvents that can be used to dissolve the compound of Formula III can be ethanol, dimethyl sulfoxide, methylene chloride, chloroform, tetrahydrofuran, acetone, dioxane, ethyl acetate, dimethylene carbonate, dimethyl formamide, methyl ethyl ketone, butyl acetate, butyl propionate, diethyl carbonate, or a mixture of any two or more solvents provided above. In a preferred embodiment, the solvent is a mixed solvent. In a particularly preferred embodiment, the solvent is a mixed solvent containing ethanol and acetone, wherein the ratio of ethanol to acetone is within the range between 1/1 (v/v) and 10/1 (v/v), between 1/1 (v/v) and 8/1 (v/v), between 1/1 (v/v) and 6/1 (v/v), between 1/1 (v/v) and 4/1 (v/v), and between 1/1 (v/v) and 2/1 (v/v). In one embodiment, the ratio of ethanol to acetone is 1/1 (v/v). The optimum amount of solvent required to dissolve the compound of Formula III will be dependent upon the amount of Formula III, reaction condition, and the purification procedure. In a particularly preferred embodiment, about 180 mL ethanol/acetone is used to dissolve about 8 grams of the compound of Formula III.

In some embodiments, step (b) of the methods includes sub-steps, including:

(b1) providing a reaction temperature between −20° C. and 10° C.;

(b2) adding an acid to the reaction mixture;

(b3) adding an oxidizing agent to the reaction mixture; and

(b4) adding a base to the reaction mixture.

It has been discovered that the reaction temperature and/or the step (b4) is important in producing the compound of Formula II with higher purity. Failure to maintain the temperature within the range disclosed herein and/or failure to perform step (b4) leads to undesired polymerization of the compounds of Formula II, thus introduce impurities. For example, a reaction temperature higher than 5° C. can cause formation of byproducts or polymerization of the product. The reaction temperature is provided and maintained within the range between −20° C. and 10° C., between −20° C. and 5° C., between −20° C. and 0° C., between −15° C. and 10° C., between −15 C and 5° C., and between −15° C. and 0° C., or less than 5° C. In one embodiment, the reaction temperature is provided and maintained between −15° C. and 5° C. In a preferred embodiment, the reaction temperature is provided and maintained at −15° C. In some embodiments, under low reaction temperature (e.g. −15° C.), the reaction mixture (e.g., reaction solution) may get icy that stops magnetic stirring; mechanic stirring could be used in these instances. It is discovered that slow stirring or null stirring does not affect the outcome of the reaction.

The acid added to the reaction mixture can be hydrochloric acid (HCl), nitric acid, hydroiodic acid, perchloric acid, chloric acid, sulfuric acid, hydrobromic acid, sulfurous acid, methanoic acid, phosphoric acid, nitrous acid, oxalic acid, methanoic acid, benzoic acid, acetic acid, formic acid, or hydrofluoric acid. In a preferred embodiment, the acid is HCl.

In some embodiments, the concentration of the acid is between 2 M and 10 M, between 3 M and 10 M, between 4 M and 10 M, between 5 M and 10 M, between 2 M and 9 M, between 3 M and 9 M, between 4 M and 9 M, between 5 M and 9 M, between 2 M and 8 M, between 3 M and 8 M, between 4 M and 8 M, between 5 M and 8 M, between 2 M and 7 M, between 3 M and 7 M, between 4 M and 7 M, between 5 M and 7 M, between 2 M and 6 M, between 3 M and 6 M, between 4 M and 6 M, between 5 M and 6 M, between 4.5 M and 6 M, or between 4.5 M and 5.5 M. In some embodiments, the concentration of the acid is about 5 M. In a particular embodiment, the concentration of HCl is about 5 M. Failure to use the concentration of HCl disclosed herein can result in insufficient removal of impurities and/or undesirable polymerization of the compound of Formula II.

The oxidizing agent added to the reaction mixture can be hydrogen peroxide and inorganic peroxides, nitric acid and nitrate compounds, sulfuric acid, peroxydisulfuric acid, persulfate compounds such as ammonium persulfate and sodium persulfate, peroxymonosulfuric acid, permanganate compounds such as potassium permanganate, sodium perborate, or potassium nitrate. In a preferred embodiment, the oxidizing agent is sodium persulfate. In some other preferred embodiments, the oxidizing agent is ammonium persulfate. Generally, the oxidizing agent is less than 4 equivalent (mole) to the compound of Formula III, such as about 1 equivalent (mole), 2 equivalent (mole), 3 equivalent (mole) to the compound of Formula III, preferably 1 equivalent (mole) to the compound of Formula III. The oxidizing agent may be added to the reaction mixture over a period between 5 and 300 seconds, between 5 and 200 seconds, between 5 and 100 seconds, between 5 and 60 seconds, between 5 and 50 seconds, between 5 and 40 seconds, between 5 and 30 seconds, between 10 and 60 seconds, between 10 and 50 seconds, between 10 and 40 seconds, between 20 and 60 seconds, between 20 and 50 seconds, and between 20 and 40 seconds. In one embodiment, the oxidizing agent is added to the reaction mixture over a period of 30 seconds.

The base is added to the reaction mixture to quench the reaction. The base can be sodium hydroxide (NaOH), lithinium hydroxide (LiOH), potassium hydroxide (KOH), rubidium hydroxide (RbOH), or cesium hydroxide (CsOH). In a preferred embodiment, the base is NaOH. In some embodiment, the base-added reaction mixture is vigorously mixed for a period between 1 and 30 minutes, between 1 and 20 minutes, between 1 and 10 minutes, between 1 and 5 minutes, and between 1 and 3 minutes. In one embodiment, the base-added reaction mixture is vigorously mixed for 2 minutes. In some embodiments, the mixing step can quench the acid for full neutralization.

In some embodiments, step (c) includes collecting the compound of Formula II and washing with water. In a preferred embodiment, the washing temperature should be less than 50° C. and greater than 0° C., and more preferably at room temperature. The composition containing the compound of Formula II may be collected by any suitable means. In some embodiment, the composition is collected by filtration or centrifugation. In a particular embodiment, the reaction mixture is filtered using a 600 mL (40-60 C) frit sintered glass filter funnel equipped with grade 415 filter paper (qualitative, grade 415 was used (VWR®)).

Although it is not necessary to completely dry the compound of Formula II after washing, in a preferred embodiment, the washed compound of Formula II is dried by any suitable means. In some embodiments, the compound of Formula II is air-dried, dried in a vacuum oven, or using a dehydrating agent. In a preferred embodiment, the compound of Formula II is dried in a vacuum oven at a temperature of about 80° C. for about 12 hours.

C. Methods of Making the Composition Containing Compound of Formula I

The compound of Formula I is made using the compound of Formula III as the starting material using the steps of:

(a) dissolving a compound of Formula III in a solvent to provide a reaction mixture;

(b) providing a reaction condition for the reaction mixture to form the compound of Formula I; and

(c) purifying the compound of Formula I from the reaction mixture to give the composition containing the compound of Formula I.

The purification step (c) of the methods does not require complicated purification techniques, such as liquid chromatograph. This is particularly desirable since it greatly simplifies the purification process and lowers the cost of synthesis.

The resulting composition has between 90% and 99% by weight the compound of Formula I, preferably between 95% and 99% by weight the compound of Formula I, more preferably between 98% and 99% by weight the compound of Formula I.

The methods result in a yield of compound of Formula I between 80% and 99%, between 85% and 99%, and between 90% and 99% by weight. In a preferred embodiment, the compound of Formula I has a yield between 92% and 97% by weight.

(a) Dissolving a Compound of Formula III in a Solvent to Provide a Reaction Mixture

The amount of the compound of Formula III in step (a) used as starting material is preferably between 5 and 100 grams, between 5 and 80 grams, between 5 and 50 grams, between 5 and 20 grams, and between 5 and 10 grams. In one embodiment, the compound of Formula III used as starting material about 8 grams.

Example solvent to dissolve the compound of Formula III can be ethanol, dimethyl sulfoxide, methylene chloride, chloroform, tetrahydrofuran, acetone, dioxane, ethyl acetate, dimethylene carbonate, dimethyl formamide, methyl ethyl ketone, butyl acetate, butyl propionate, diethyl carbonate, or a combination thereof. In a preferred embodiment, the solvent is a mixed solvent containing two or more the afore-mentioned solvents. In a particularly preferred embodiment, the solvent is a mixed solvent containing ethanol and acetone, wherein the ratio of ethanol to acetone is within the range between 1/1 (v/v) and 10/1 (v/v), between 1/1 (v/v) and 8/1 (v/v), between 1/1 (v/v) and 6/1 (v/v), between 1/1 (v/v) and 4/1 (v/v), and between 1/1 (v/v) and 2/1 (v/v). In one embodiment, the ratio of ethanol to acetone is 1/1 (v/v). The optimum amount of solvent required to dissolve the compound of Formula III will be dependent upon the amount of Formula III, reaction condition, and the purification procedure. In a particularly preferred embodiment, about 30 mL ethanol/acetone is used to dissolve about 8 grams of the compound of Formula III.

(b) Reaction Conditions for the Reaction Mixture to Form the Compound of Formula I

Conditions to form the compound of formula I include:

(b1) providing a reaction temperature between −20° C. and 10° C.;

(b2) adding an acid to the reaction mixture;

(b3) adding the reaction mixture to a solution comprising an oxidizing agent to give a second mixture; and

(b4) adding a base to the second reaction mixture.

It has been discovered that the reaction temperature and/or step (b4) is critical in producing the compound of Formula II with higher purity. Failure to maintain the temperature within the range disclosed herein and/or failure to perform step (b4) leads to undesired polymerization of the compounds of Formula I, thus introduce impurities. The reaction temperature is provided and maintained within the range between −20° C. and 10° C., between −20° C. and 5° C., between −20° C. and 0° C., between −15° C. and 10° C., between −15 C and 5° C., and between −15° C. and 0° C. In one embodiment, the reaction temperature is provided and maintained between −15° C. and 5° C. In a preferred embodiment, the reaction temperature is provided and maintained at −15° C.

The acid added to the reaction mixture can be hydrochloric acid (HCl), nitric acid, hydroiodic acid, perchloric acid, chloric acid, sulfuric acid, hydrobromic acid, sulfurous acid, methanoic acid, phosphoric acid, nitrous acid, oxalic acid, methanoic acid, benzoic acid, acetic acid, formic acid, or hydrofluoric acid. In a preferred embodiment, the acid is acetic acid. After adding the acid, the reaction mixture is mixed for a period between 0.1 and 5 hours to ensure complete dissolution of the compound of Formula III in acidic conditions. In some embodiments, the reaction mixture is mixed for a period between 0.1 and 5 hours, between 1 and 5 hours, between 1 and 4 hours, between 1 and 3 hours. In a particular embodiment, the reaction mixture is mixed for about 2 hours by stirring.

The oxidizing agent can be hydrogen peroxide and inorganic peroxides, nitric acid and nitrate compounds, sulfuric acid, peroxydisulfuric acid, persulfate compounds such as sodium persulfate and ammonium persulfate, peroxymonosulfuric acid, permanganate compounds such as potassium permanganate, sodium perborate, or potassium nitrate. In a preferred embodiment, the oxidizing agent is sodium persulfate. In some other preferred embodiments, the oxidizing agent is ammonium persulfate. Generally, the oxidizing agent is at least 4 equivalent (mole) to the compound of Formula III, such as about 4 equivalent (mole), about 5 equivalent (mole), about 6 equivalent (mole), about 7 equivalent (mole), about 8 equivalent (mole), about 9 equivalent (mole), or about 10 equivalent (mole) to the compound of Formula III.

It has been discovered that the order of step (b3) and the amount of the reaction mixture added to the solution containing an oxidizing agent are important to avoid polymerization during the reactions. It is important to add the reaction mixture to the solution containing an oxidizing agent to avoid undesirable polymerization of the compounds of Formula I. Preferably, the reaction mixture added to the solution containing an oxidizing agent does not exceed about 50 mL, about 40 mL, 30 mL, 20 mL, or 10 mL, and is between 1/10 and 1/100 (v/v) of the solution containing the oxidizing agent. In a preferred embodiment, the reaction mixture added to the solution containing an oxidizing agent is about 1/50 (v/v) of the solution containing an oxidizing agent. In the most preferred embodiment, 20 mL of the reaction mixture is added to 1 L of the solution containing sodium persulfate. In some embodiments, the reaction mixture provided in step (b2) is more than 200 mL in volume, wherein step (b3) can be performed by dividing the reaction mixture in small portions with between about 10 mL and about 40 mL, between about 10 mL and about 30 mL, between about 10 mL and about 20 mL, between about 20 mL and about 40 mL, or between about 30 mL and about 40 mL per portion and repeat steps (b3) and (b4) with each portion until all the reaction mixture are used.

The base is added to the second reaction mixture to quench the reaction. The base can be sodium hydroxide (NaOH), lithinium hydroxide (LiOH), potassium hydroxide (KOH), rubidium hydroxide (RbOH), or cesium hydroxide (CsOH). In a preferred embodiment, the base is NaOH. It has been discovered that the amount of base added to quench the reaction is critical to avoid polymerization and result in pure compounds of Formula I. Addition of large amount of base to the second reaction mixture generates heat, which leads to polymerization of the compound of Formula I. Preferably, the base is added to the second reaction mixture in portions, wherein each portion is about 1/1 (v/v) of the reaction mixture added to the solution containing an oxidizing agent in step (b3). At least 4 portions of the base is added to the second reaction mixture. It has been discovered that excess amounts of water, oxidizing agent, and base do not affect the reaction.

(c) Purifying the Compound of Formula I

Step (c) includes collecting the compound of Formula I from the reaction mixture and washing the compound of Formula I with water. The temperature of washing the compound of Formula I should be less than 50° C. and greater than 0° C., and more preferably at room temperature. The composition containing the compound of Formula I may be collected by any suitable means. In some embodiment, the compound of Formula I is collected by filtration or centrifugation. In a particular embodiment, the reaction mixture is filtered using a 600 mL (40-60 C) frit sintered glass filter funnel equipped with grade 415 filter paper (qualitative, grade 415 was used (VWR®)).

Although it is not necessary to completely dry the compound of Formula I after washing, in a preferred embodiment, the washed compound of Formula I is air-dried or dried using a dehydrating agent. It has been discovered that the heat of a vacuum oven promotes decomposition of the compound of Formula I and impurities will be introduced. In a preferred embodiment, the compound of Formula I is dried using a dehydrating agent between 12 and 24 hours. The dehydrating agent can be aluminum phosphate, methyl N-(triethylammoniumsulfonyl)carbamate, calcium oxide, cyanuric chloride, N,N′-Dicyclohexylcarbodiimide, iron(III) chloride, orthoformic acid, phosphorus pentoxide, phosphoryl chloride, sulfuric acid, or a combination thereof. In a preferred embodiment, the washed compound of Formula I is dried using phosphorus pentoxide (P₂O₅) for about 24 hours.

### IV. Methods of Using

The disclosed compositions containing oligoanilines can be sued in dye applications as dye molecules. In some embodiments, the compositions of oligoanilines are redox active dye molecules that undergo a color change during a reduction/oxidation reaction (redox reaction). This color change is indicative of the progress and/or the completion of the redox reaction. Generally, the color change is reversible and is dependent upon the reduction-oxidation equilibrium of the oligoaniline compound. The oligoaniline compositions may be added in a test solution or immobilized on a surface by any suitable means such as drop casting or spin coating and brought in contact with a test solution. One or more compositions of oligoanilines may be used in an array in different test solutions. The one or more compositions of oligoanilines may have the same color or different colors.

In a preferred embodiment, the compound of Formula III is N¹,N⁴-Bis(4-aminophenyl)-1,4-benzenediamine, in which R₁-R₈ are all hydrogens and m=1, n=1. The compound, N¹,N⁴-Bis(4-aminophenyl)-1,4-benzenediamine has a white color. The composition containing N¹,N⁴-Bis(4-aminophenyl)-1,4-benzenediamine has a lavender color because of insignificant amount (≤2% by weight, determined by elemental analysis) of oxidation byproduct. This composition can be used as a reducing agent in a redox reaction and its color change from lavender to dark blue being indicative of the progress and/or completion of the redox reaction. The color change can be reversible and is dependent upon the reduction-oxidation equilibrium of N¹,N⁴-Bis(4-aminophenyl)-1,4-benzenediamine during the redox reaction.

In a preferred embodiment, the compound of Formula II is N¹,N⁴-Bis(4-aminophenyl)-1,4-quinonediimine, in which R₁-R₈ are all hydrogens and m=1, n=1. The compound, N¹,N⁴-Bis(4-aminophenyl)-1,4-quinonediimine has a white color. The composition containing N¹,N⁴-Bis(4-aminophenyl)-1,4-quinonediimine has a blue color. This composition can be used as an oxidizing agent in a redox reaction and its color change from blue to white is indicative of the progress and/or completion of the redox reaction. This composition can be used as an reducing agent in a redox reaction and its color change from blue to red is indicative of the progress and/or completion of the redox reaction. The color change can be reversible and is dependent upon the reduction-oxidation equilibrium of N¹,N⁴-Bis(4-aminophenyl)-1,4-quinonediimine during the redox reaction.

In a preferred embodiment, the compound of Formula I is N¹,N⁴-(1,4-phenylene)bis(quinonediimine), in which R₁-R₈ are all hydrogens and m=1, n=1. The compound, N¹,N⁴-(1,4-phenylene)bis(quinonediimine) has a red color. The composition containing N¹,N⁴-(1,4-phenylene)bis(quinonediimine) has a red color. This composition can be used as an oxidizing agent in a redox reaction and its color change from red to blue is indicative of the progress and/or completion of the redox reaction. The color change can be reversible and is dependent upon the reduction-oxidation equilibrium of N¹,N⁴-(1,4-phenylene)bis(quinonediimine) during the redox reaction. In some embodiments, the compound, N¹,N⁴-(1,4-phenylene)bis(quinonediimine), can oxidize alcohols to result in aldehydes. In some embodiments, the compound, N¹,N⁴-(1,4-phenylene)bis(quinonediimine), can oxidize water.

The compositions disclosed herein can also be used as intermediates for the development of conductive elastomers, or as catalysts

The present invention will be further understood by reference to the following non-limiting examples.

### EXAMPLES

The Examples demonstrate the synthesis of highly pure oligoanilines in large scale. The Examples also show the synthesis of a new oxidized oligoanilines such as compound of Formula I. The synthesis provided herein demonstrates control of reaction temperatures, reagents and purification steps that are critical to avoid polymerization during reactions to yield pure oligoaniline products. Such synthesis does not require complicated purification technique such as liquid chromatography, can be performed in large scale, e.g. starting materials in grams, and is characterized by high yield (90-97%) and high purity (90-99%). The products obtained can be fully characterized by NMR, Mass spectrometry, and Elemental analysis. The disclosed large scale synthesis of oligoanilines represents an easier access to oligoanilines and expedites the discovery of additional properties and/or applications of oligoanilines.

### Example 1. Synthesis of N¹,N⁴-Bis(4-nitrophenyl)-1,4-benzenediamine (3)

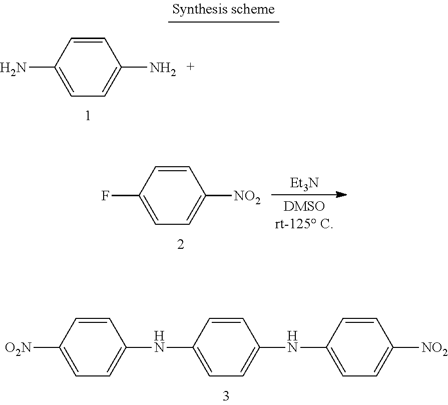

An oven-dried, 1-L 24/40 round-bottomed flask, open to air, and equipped with a 5-cm egg-shaped, Teflon-coated, magnetic stir bar. 12.3 g (0.114 mol, 1.0 equiv.) of p-phenylenediamine (1) (99%, purchased from MilliporeSigma and used as received), 40 g (33 mL, 0.285 mol, 2.5 equiv.) of 1-fluoro-4-nitrobenze (2) (98%, purchased from Oakwood Chemical and used as received), 200 mL of dimethyl sulfoxide (DMSO) (99%, purchased from MilliporeSigma and used as received), and 23 g (32 mL, 0.228 mol, 2.0 equiv.) of triethylamine (Et₃N) (99%, purchased from Acros Organics was used as received) was added in the flask. A reflux condenser was connected to the round-bottomed flask. The condenser was required because the boiling point of Et₃N (bp=89° C.) is lower than reaction temperature, and any condenser can be used.

The reaction mixture was placed in an oil bath at ambient temperature and the temperature was slowly increased to 125° C. and stirred for 3 days. After 3 days, the reaction mixture was allowed to cool down to room temperature. Then, 20 mL of dichloromethane (DCM) (purchased from Fisher Scientific Chemical and used as received) was added to the dark red reaction mixture and stirred for about 10 min. 5 M hydrochloric acid (HCl) was prepared using deionized water and concentrated hydrochloric acid (purchased from Macron Fine Chemicals). 400 mL of the 5 M HCl was added to the reaction mixture while stirring. The heated up reaction mixture during the addition of HCl was allowed to cool down to room temperature.

The formed dark precipitate was filtered using a 600 mL (40-60 C) frit sintered glass filter funnel equipped with grade 415 filter paper (qualitative, grade 415 was used (VWR®)). The round-bottomed flask was rinsed with 50 mL of 5 M HCl/DMSO (2/1, v/v) solution for three times (3×50 mL), which was transferred into the frit sintered glass funnel. The red solid in the filter was washed with deionized water for three times (3×100 mL).

To remove the side products in the reaction mixture, 100 mL of 5 M HCl/DMSO (2/1, v/v) was added to the red solid in the filter funnel followed by washing with deionized water twice (2×100 mL). This process was repeated for 6 additional times with a total of 700 mL of 5 M HCl/DMSO (2/1, v/v) solution and 1400 mL of deionized water to remove side products, which are soluble in water under acidic conditions. To ensure neutralization, the solid was further washed with deionized water for 5 times (5×100 mL). The red solid was kept in the glass filter funnel under vacuum suction for 1 h to remove excess water. Finally, the solid was washed with dichloromethane (DCM) for 5 times (5×100 mL).

The red solid was dried using a vacuum oven (80° C./ca. 25 Torr) for about 12 hrs to give 31.5-32.7 g (79-82% yield) of pure N¹,N⁴-Bis(4-nitrophenyl)-1,4-benzenediamine (3) as a deep red-brown solid. The purity of compound 3 is about 98% by elemental analysis.

Characterization data of 3: mp 278-280° C. ¹H NMR (DMSO, 500 MHz): δ=6.99 (d, J=9.0 Hz, 4H, ═CH), 7.23 (s, 4H, ═CH), 8.04 (d, 4H, J=9.0 Hz, ═CH), 9.26 (s, 2H, NH). ¹³C NMR (DMSO, 125 MHz): δ=113.1, 122.4, 126.2, 135.7, 137.7, 151.1. IR (neat, cm⁻¹): 3379, 3301, 1587, 1509, 1468, 1289, 1180, 1107, 853, 827, 692, 499. HRMS m/z (ESI) calculated for C₁₈H₁₅N₄O₄ [M+H]⁺ 351.1088, found 351.1091. Elemental analysis calculated for C₁₈H₁₄N₄O₄: C, 61.71; H, 4.03; N, 15.99; O, 18.27, found: C, 60.51; H, 3.76; N, 15.29.

### Example 2. Synthesis of N¹,N⁴-Bis(4-aminophenyl)-1,4-benzenediamine (4)

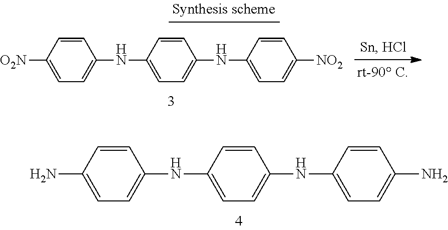 [SMILES: O=[N+]([O-])c1ccc(Nc2ccc(Nc3ccc([N+](=O)[O-])cc3)cc2)cc1 ; Nc1ccc(Nc2ccc(Nc3ccc(N)cc3)cc2)cc1]

To an oven-dried, 1-L 24/40 round-bottomed flask, open to air and equipped with a 5-cm egg-shaped, Teflon-coated, magnetic stir bar, was added 30.0 g (0.0856 mol, 1.0 equiv.) N¹,N⁴-Bis(4-nitrophenyl)-1,4-benzenediamine (3) synthesized according to the above described procedure, 50.8 g (0.428 mol, 5.0 equiv.) of granular tin (99.9%, purchased from Chem Impex and used as received), and 500 mL of concentrated hydrochloric acid (purchased from Macron Fine Chemicals). A reflux condenser was connected to the round-bottomed flask. This reaction mixture was stirred at room temperature for 2 hrs. The reaction mixture must be stirred at room temperature, for 2 hours, to prevent overheating (exothermic reaction). Overheating will cause the reaction mixture to spill over.

The reaction mixture was then placed in a silicone oil bath and the temperature is gradually increased to 90° C. (in about 20 min.). After 1 h, any material adhering to the sides of the round-bottomed flask was rinsed into the reaction mixture with small amount (about 15 mL) of concentrated HCl to ensure that all starting materials and reagents fully react (perform this after about 1 hour). The reaction mixture was stirred for an additional 5 h at 90° C. and allowed to cool down to room temperature.

The brownish precipitate was filtered using a 600 mL (40-60 C) frit sintered glass filter funnel equipped with grade 415 filter paper (qualitative, grade 415 was used (VWR®)). The round-bottomed flask was rinsed with 20 mL concentrated HCl solution for three times (3×50 mL) which was then transferred into the glass filter funnel. The crude product was washed with 5 M HCl solution for 6 times (6×50 mL) to remove any excess tin. The resulting white solid is transferred into a 500 mL beaker equipped a 5-cm egg-shaped, Teflon-coated, magnetic stir bar. To ensure complete transfer of the product, the filter funnel was washed three times with 30 mL of deionized water (3×30 mL), which are then poured into the beaker.

2.5 M sodium hydroxide solution (NaOH) was prepared using deionized water and sodium hydroxide pellets (>97.0%) purchased from Fisher Scientific (30 g of NaOH in 300 mL of deionized water). 300 mL of the 2.5 M NaOH solution was added to the beaker. The mixture is stirred for 30 min and filtered using a 600 mL (40-60 C) frit sintered glass filter funnel equipped with grade 415 filter paper (qualitative, grade 415 was used (VWR®)). The beaker was rinsed three times with 50 mL of 2.5 M NaOH solution (3×50 mL), and then transferred into the filter funnel. The solid was neutralized with deionized water (10×100 mL). The product has lavender color because of an insignificant amount of oxidation byproduct N¹,N⁴-Bis(4-aminophenyl)-1,4-quinonediimine, otherwise the color should be white.

The white solid was dried using a vacuum oven (80° C./ca. 25 Torr) for about 12 hrs to give 20.9-21.8 g (90-94% yield) of pure N¹,N⁴-Bis(4-aminophenyl)-1,4-benzenediamine (4) as a lavender solid. The purity of compound 4 is about 98% by elemental analysis.

Characterization data of 4: mp 194-196° C. ¹H NMR (DMSO, 500 MHz): δ=4.56 (s, 4H, NH2), 6.48 (d, J=8.5 Hz, 4H, ═CH), 6.71 (s, 8H, ═CH), 7.00 (s, 2H, NH). ¹³C NMR (DMSO, 125 MHz): δ=114.9, 117.0, 119.6, 134.3, 138.0, 142.1. IR (neat, cm⁻¹): 3363, 3308, 3207, 1599, 1514, 1498, 1287, 1260, 853, 735, 561, 504. HRMS m/z (ESI) calculated for C₁₈H₁₉N₄ [M+H]⁺ 291.1604, found 291.1609. Elemental analysis calculated for C₁₈H₁₈N₄: C, 74.46; H, 6.25; N, 19.30, found: C, 74.34; H, 6.29; N, 19.30.

### Example 3. Synthesis of N¹,N⁴-Bis(4-aminophenyl)-1,4-quinonediimine (5)

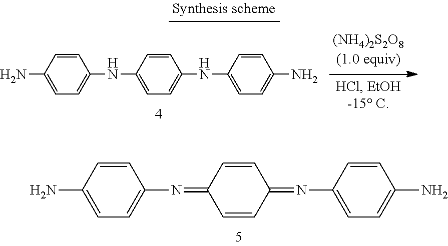 [SMILES: Nc1ccc(Nc2ccc(Nc3ccc(N)cc3)cc2)cc1 ; Nc1ccc(N=C2C=CC(=Nc3ccc(N)cc3)C=C2)cc1]

An oven-dried, 1-L 24/40 round-bottomed flask was open to air and equipped with 5-cm egg-shaped, Teflon-coated, magnetic stir bar. 8.0 g (0.027 mol) was charged with N¹,N⁴-Bis(4-aminophenyl)-1,4-benzenediamine (4) (synthesized according to the above described procedure) and 180 mL solution of ethanol/acetone (1/1, v/v)(Note 22). The mixture was stirred at room temperature for 5 minutes to completely dissolve the aniline oligomer 4.

The round bottom flask was placed in a ˜15° C. bath. The cold bath was prepared using 200 g of dry ice in 500 mL ethylene glycol (purchased from Fisher Scientific Chemical). Solution may get icy and stop stirring. The slow stirring or null stirring did not affect the outcome of the reaction. Once the reaction mixture reached −15° C. in about 5 min., 300 mL of 1 M hydrochloric acid (HCl) solution (prepared using deionized water and concentrated hydrochloric acid purchased from Macron Fine Chemicals) was added to the violet mixture. The reaction mixture was allowed to stir for another 5 minutes. 6.2 g (0.027 mol, 1.0 equiv) ammonium persulfate ((NH₄)₂S₂O₈) (>98%, purchased from Acros Organics and used as received) was added over 30 seconds while the mixture is still in the cold bath and under stirring.

A thermometer was inserted in the round bottom flask and the reaction is removed from the cold bath. As soon as the temperature inside the reaction flask reached 5° C., the reaction mixture is put back in the cold bath because the reaction product may polymerize if the temperature goes over 5° C. The reaction mixture was stirred for 10 minutes at −15° C., and 200 mL of 2.5 M of sodium hydroxide (NaOH) solution (prepared using deionized water and sodium hydroxide pellets, purchased from Fisher Scientific) was added while still in the cold bath. The reaction mixture was removed from the cold bath and vigorously stirred for 2 min; then the violet mixture was filtered under vacuum filtration using a 600 mL (40-60 C) frit sintered glass filter funnel equipped with grade 415 filter paper (qualitative, grade 415 was used (VWR®)). The round-bottomed flask was rinsed twice with 20 mL solution of 2.5 M NaOH solution (2×20 mL) and poured into the frit sintered glass filter funnel. The blue solid was washed with deionized water (3×100 mL) and kept in the glass filter funnel under vacuum suction for 20 min. to remove excess of water.

Finally, the blue solid was dried using a vacuum oven (80° C./ca. 25 Torr) for about 12 hrs to give 7.2-7.6 g (91-96% yield) of pure N¹,N⁴-Bis(4-aminophenyl)-1,4-quinonediimine (5) as a dark blue solid. The purity of compound 5 was about 98% by elemental analysis.

Characterization data of 5: mp 168-170° C. ¹H NMR (DMSO, 500 MHz): δ=5.43 (s, 4H), 6.60-6.79 (m, 4H), 6.89-7.05 (m, 8H). ¹³C NMR (DMSO, 125 MHz): δ=114.0, 123.0, 124.1, 124.3, 135.2, 136.8, 139.2, 139.3, 147.6, 147.8, 155.1. IR (neat, cm⁻¹): 3379, 3309, 3206, 1630, 1542, 1318, 1166, 984, 830, 699, 541, 506, 411. HRMS m/z (ESI) calculated for C₁₈H₁₇N₄[M+H]⁺ 289.1448, found 289.1443. Elemental analysis calculated for C₁₈H₁₆N₄: C, 74.98; H, 5.59; N, 19.43, found: C, 73.88; H, 6.08; N, 18.94.

### Example 4. Synthesis of N¹,N⁴-(1,4-Phenylene)bis(quinonediimine) (6)

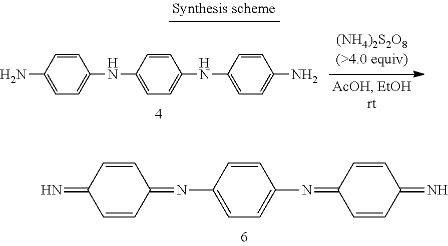 [SMILES: Nc1ccc(Nc2ccc(Nc3ccc(N)cc3)cc2)cc1 ; N=C1C=CC(=Nc2ccc(N=C3C=CC(=N)C=C3)cc2)C=C1]

An oven-dried, 1 L 24/40 round-bottomed flask was open to air and equipped with 5-cm egg-shaped, Teflon-coated, magnetic stir bar. 8.0 g (0.027 mol, 1.0 equiv) N¹,N⁴-Bis(4-aminophenyl)-1,4-benzenediamine (4) synthesized according to the above described procedure and 30 mL of ethanol/acetone (1/1, v/v, prepared from ethanol (99.5%) and acetone (>99.5%), both purchased from Fisher Scientific Chemicals and used as received) solution are added into the flask. The mixture was stirred at room temperature for 20 min. 200 mL of glacial acetic acid (purchased from Millipore Sigma was used as received) was added to the violet mixture and allowed to stir for 2 h to ensure complete dissolution.

In a separate 2-L 24/40 round-bottomed flask equipped with 5-cm egg-shaped, Teflon-coated, magnetic stir bar, an ammonium persulfate solution was prepared with 8.0 g (0.035 mol, 1.3 equiv) of ammonium persulfate in 1 liter of deionized water under stirring for about 1 min at room temperature to ensure dissolution. To this ammonium persulfate solution under stirring, 20 mL of the aniline trimer 4 mixture was added and stirred for 10 seconds. The reaction mixture was then quenched with 5 M NaOH solution in portions (4×20 mL) and stirred for 5 min. More than 4 equivalents of ammonium persulfate are needed to ensure oxidation aniline trimer (4).

The red precipitate was filtered under vacuum filtration using a 600 mL (40-60 C) frit sintered glass filter funnel equipped with grade 415 filter paper (qualitative, grade 415 was used (VWR®)). Right after the filtration (without cleaning of the 2 L 24/40 round-bottomed flask), anther fresh ammonium persulfate solution was prepared by adding 8 g of ammonium persulfate in 1 L of deionized water. To this fresh ammonium persulfate solution, 20 mL of aniline trimer 4 mixture was added and stirred for 10 seconds. The reaction mixture was then quenched with 5 M NaOH solution in portions (4×20 mL) and stirred for 5 min. The red precipitate was filtered under vacuum suction filtration as described above. This process was repeated until all the aniline trimer mixture solution is used (a total of 12 times). If less than 20 mL of the trimer solution was left for the last run before oxidation, proportional amounts or ratios can be calculated. Excess amounts of reagents, water, (NH₄)₂S₂O₈, and NaOH will not affect the reaction. However, if the entire portion of the aniline trimer 4 mixture containing 200 mL glacial acetic acid solution was added to a proportional amount of ammonium persulfate in water, polymerization will be observed, even after neutralization with NaOH. Further, addition of large amount of NaOH to the reaction mixture generates heat, which leads to polymerization of the aniline trimers. It is important to add NaOH in portions to the reaction mixture.

The 2 L round bottom flask was rinsed twice with 40 mL of deionized water (2×40 mL), and then transferred into the frit sintered glass filter funnel. The red solid is neutralized with deionized water (5×100 mL).

The red solid was dried over 50 g of phosphorous pentoxide (P₂O₅) (purchased from J T Baker and used as received) under reduced pressure (25 Torr) for about 24 h to give 7.2-7.6 g (92-97%) of pure N¹,N⁴-(1,4-Phenylene)bis(quinonediimine) (6), as a red solid. The purity of compound 6 is about 98% by elemental analysis. The products cannot be dried in a vacuum oven because heat may promote a decomposition reaction and impurities will be observed.

Characterization data of 6: mp 149-151° C. ¹H NMR (DMSO, 500 MHz): δ=6.77-6.99 (m, 12H), 11.28 (s, 2H). ¹³C NMR (DMSO, 125 MHz): δ=121.5, 122.8, 124.9, 132.2, 133.2, 134.8, 135.8, 136.8, 137.2, 146.9, 157.4, 165.7, 165.8. IR (neat, cm⁻¹): 3180, 3051, 1623, 1577, 1160, 845, 575, 418. HRMS m/z (ESI) calculated for C₁₈H₁₅N₄ [M+H]⁺ 287.1291, found 287.1296. Elemental analysis calculated for C₁₈H₁₄N₄: C, 75.50; H, 4.93; N, 19.57, found: C, 74.85; H, 4.86; N, 19.15.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A compound of Formula I: 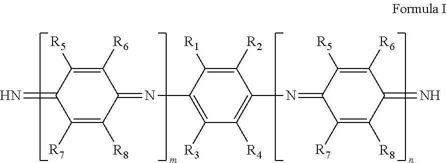
R₁ to R₈ independently represent a hydrogen atom, a halogen atom, a hydroxyl group, an amino group, a silanol group, a sulfonyl group, a thiol group, a carboxyl group, a phosphate group, a phosphoric acid ester group, an ester group, a carbonate ester group, a thioester group, an amide group, a nitro group, a monovalent hydrocarbon group, an organoxy group, an aminooxy group, a hydroxyamino group, an azo group, an organosilyl group, an organothio group, an alkyl group, an acryl group, an acyl group, an acryl group, or a sulfone group;
wherein R₁ to R₈ optionally form a ring with its neighboring R group, respectively; and
wherein m and n are independently an integer ≥1 provided that m+n≤20 is satisfied.

2. The compound of claim 1, wherein: (i) R₁ to R₈ are all hydrogen atoms or (ii) the compound is a trimer, in which m=1, n=1.

3. A composition comprising a compound selected from the group consisting of a compound of Formula I, 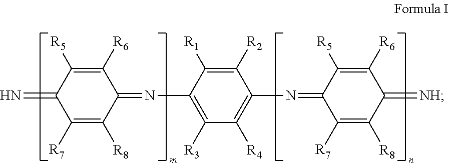
a compound of Formula II 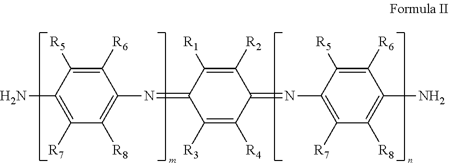
and a compound of Formula III 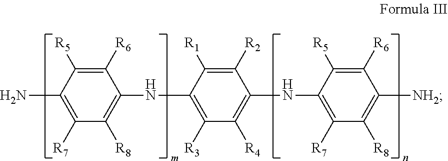
the composition having between 90% and 99% by weight the compound of Formula I,
wherein:
R₁ to R₈ independently represent a hydrogen atom, a halogen atom, a hydroxyl group, an amino group, a silanol group, a sulfonyl group, a thiol group, a carboxyl group, a phosphate group, a phosphoric acid ester group, an ester group, a carbonate ester group, a thioester group, an amide group, a nitro group, a monovalent hydrocarbon group, an organoxy group, an aminooxy group, a hydroxyamino group, an azo group, an organosilyl group, an organothio group, an alkyl group, an acryl group, an acyl group, an acryl group, or a sulfone group;
wherein R₁ to R₈ optionally form a ring with its neighboring R group, respectively; and
wherein m and n are independently an integer ≥1 provided that m+n≤20 is satisfied.

4. The composition of claim 3, wherein: (i) R₁ to R₈ are all hydrogen atoms or (ii) the compound is a trimer, in which m=1, n=1.

5. A method of making the Formula III oligoanaline of claim 3, the method comprising:
(a) reducing a compound of Formula A to provide a composition comprising the oligoaniline of Formula III; and
(b) purifying the oligoaniline of Formula III from the composition 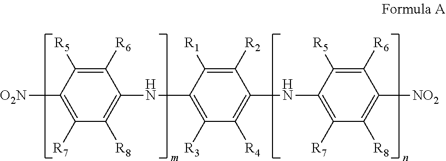
R₁ to R₈ independently represent a hydrogen atom, a halogen atom, a hydroxyl group, an amino group, a silanol group, a sulfonyl group, a thiol group, a carboxyl group, a phosphate group, a phosphoric acid ester group, an ester group, a carbonate ester group, a thioester group, an amide group, a nitro group, a monovalent hydrocarbon group, an organoxy group, an aminooxy group, a hydroxyamino group, an azo group, an organosilyl group, an organothio group, an alkyl group, an acryl group, an acyl group, an acryl group, or a sulfone group;
wherein R₁ to R₈ optionally form a ring with its neighboring R group, respectively; and
wherein m and n are independently an integer ≥1 provided that m+n≤20 is satisfied.

6. The method of claim 5, (i) wherein the oligoaniline of Formula III has a yield between 80% and 99% by weight or (ii) wherein, optionally, the compound of Formula A is synthesized by a method comprising:
(a) mixing a compound of Formula 1 with a compound of Formula 2 and an amine of Formula 3 in a solvent to provide a reaction mixture;
(b) providing a reaction condition for the reaction mixture to form the compound of Formula A; and
(c) purifying the compound of Formula A from the reaction mixture. 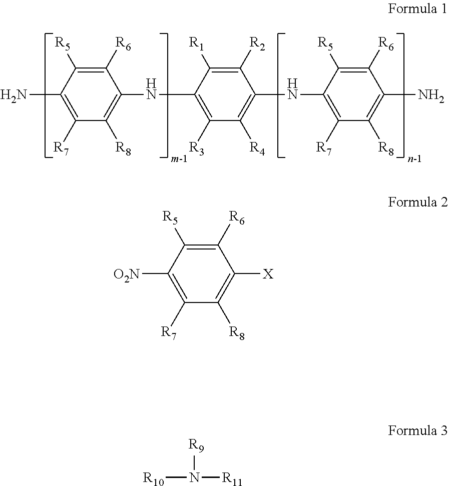
R₁ to R₈ independently represent a hydrogen atom, a halogen atom, a hydroxyl group, an amino group, a silanol group, a sulfonyl group, a thiol group, a carboxyl group, a phosphate group, a phosphoric acid ester group, an ester group, a carbonate ester group, a thioester group, an amide group, a nitro group, a monovalent hydrocarbon group, an organoxy group, an aminooxy group, a hydroxyamino group, an azo group, an organosilyl group, an organothio group, an alkyl group, an acryl group, an acyl group, an acryl group, or a sulfone group,
wherein R₁ to R₈ optionally form a ring with its neighboring R group, respectively; and
wherein R₉ to R₁₁ independently represent a monovalent hydrocarbon group,
wherein X represents a halogen, and
wherein m and n are independently an integer ≥1 provided that m+n≤20 is satisfied.

7. The method of claim 6, wherein: (i) the compound of Formula A has a yield between 70% and 99% by weight; (ii) the compound of Formula 1 is p-Phenylenediamine; (iii) the compound of Formula 2 is 1-fluoro-4-nitrobenzene; (iv) the amine of Formula 3 is triethylamine; or (iv) step (2) comprises providing a reaction temperature between 80° C. and 200° C. and a reaction time between 1 and 100 hours.

8. The method of claim 7, wherein the reaction temperature is about 125° C.; or the reaction time is about 72 hours.

9. The method of claim 5, wherein step (a) comprises:
(a1) mixing a compound of Formula A with an acid and a metal to provide a reaction mixture;
(a2) providing a reaction condition for the reaction mixture to form the compound of Formula III;

10. The method of claim 9, wherein: (i) the acid is HCl; the metal is a post-transition metal; or (iii) step (a2) comprises providing a reaction temperature between 60° C. and 100° C. and a reaction time between 1 and 100 hours.

11. The method of claim 10, wherein the reaction temperature is about 90° C.; and/or the reaction time is about 5.5 hours.

12. A method of making an oligoaniline of Formula II or Formula I, the method comprising:
(a) dissolving a compound of Formula III in a solvent to provide a reaction mixture;
(b) providing a reaction condition for the reaction mixture to form a composition comprising the oligoaniline; and
(c) purifying the oligoaniline from the composition.

13. The method of claim 12, wherein: (i) the solvent is a mixed solvent; or (iii step (b) comprises:
(b1) providing a reaction temperature between −20° C. and 10° C.;
(b2) adding an acid to the reaction mixture;
(b3) adding an oxidizing agent to the reaction mixture; and
(b4) adding a base to the reaction mixture.

14. The method of claim 13, wherein the mixed solvent comprises ethanol and acetone

15. The method of claim 13, wherein the reaction temperature is about −15° C.

16. The method of claim 13, wherein the acid is HCl.

17. The method of claim 13, wherein the acid is acetic acid.

18. The method of claim 13, wherein the oxidizing agent is ammonium persulfate.

19. The method of claim 13, wherein the base is NaOH.

20. The method of claim 12, wherein the oligoaniline has a yield between 80% and 99% by weight.
